(19) United States
(12) Patent Application Publication
(10) Pub. No.: US 2026/0137942 A1
(43) Pub. Date: May 21, 2026

(54) SYSTEMS AND METHODS FOR ONSET-FREE CONDUCTION BLOCK

(71) Applicant: DUKE UNIVERSITY, Durham, NC (US)

(72) Inventors: Warren GRILL, Durham, NC (US); Edgar PENA, Durham, NC (US); Nicole PELOT, Durham, NC (US)

(21) Appl. No.: 19/118,417

(22) PCT Filed: Oct. 10, 2023
(86) PCT No.: PCT/US2023/034811

§ 371 Date: Apr. 4, 2025

Related U.S. Application Data
(60) Provisional application No. 63/414,623, filed on Oct. 10, 2022.

(51) Int. Cl. A61N 1/36

(52) U.S. Cl. CPC A61N 1/36071; A61N 1/36192; A61N 1/36196

(57) ABSTRACT

The present disclosure provides systems and methods relating to neuromodulation. In particular, the present disclosure provides systems and methods for eliminating the onset response when blocking neural conduction. The various embodiments disclosed herein include methods and systems for delivering treatment based on blocking waveforms to subjects with pathological neural activity.

### RELATED APPLICATIONS

This application claims priority to and the benefit of U.S. Provisional Patent Application No. 63/414,623 filed Oct. 10, 2022, which is incorporated herein by reference in its entirety for all purposes.

### GOVERNMENT FUNDING

This invention was made with government support under Federal Grant No. OT2 OD025340 and R01 NS126376 awarded by the National Institutes of Health. The Federal Government has certain rights to the invention.

### FIELD

The present disclosure provides systems and methods relating to neuromodulation. In particular, the present disclosure provides systems and methods for eliminating the onset response when blocking neural conduction.

### BACKGROUND

Many neurological disorders, including pain and spasticity, are characterized by undesirable increases in sensory, motor, or autonomic nerve activity. Local application of kilohertz frequency alternating currents (KHFAC) can effectively and completely block the conduction of undesired hyperactivity through neurons and could be a therapeutic approach for alleviating disease symptoms. However, KHFAC signals produce an undesirable onset response, i.e., kilohertz frequency-induced excitation (evoked action potentials) prior to producing neural block, termed “onset response”. This onset response may result in muscle contraction and pain or other undesirable physiological response and is a significant impediment to potential clinical applications of KHFAC neural block. There are some existing approaches to reduce the onset response, such as electrode geometry design, and amplitude and frequency modulation of the applied signal. However, these approaches are unable to eliminate the onset firing and can be challenging for clinical implementation. Hence, there is an ongoing need for improved methods of KHFAC treatment.

Reversible block of neural conduction using kilohertz-frequency (KHF) electrical signals has substantial potential for the treatment of disease. KHF neural block enables a form of neural control that is complementary to neural stimulation or activation: KHF signals can block neural conduction almost immediately upon KHF signal onset, and neural conduction returns almost immediately upon KHF cessation after brief applications of KHF signals. However, onset response, i.e., KHF-induced excitation that occurs prior to producing neural block, is an undesired outcome of neural block protocols, and previous studies of KHF neural block observed onset response. The presence of and variable intensity and duration of this onset response are potential obstacles for clinical translation of neural block.

Prior studies of slowly increasing the amplitude (ramping) of the block signal reported longer and larger onset responses, but used a limited range of ramp durations (up to 60 s) and a limited range of kilohertz frequency block signals (up to 30 kHz). While hybrid waveforms that used charge imbalances could eliminate onset response, the use of charge-balanced waveforms is preferable due to the risk of damage to the electrode or tissue from charge imbalance. Thus, there is an ongoing need for systems and methods that block without producing onset response and without charge imbalances.

One previous study evaluated the ability of linearly ramped KHF signals to eliminate onset response, but it found that ramping made onset response longer and more intense (Miles et al., 2007). Such findings inform our fundamental understanding of neural response to KHF signals, which states that KHF signals produce no responses at low amplitudes, produce excitation at intermediate amplitudes, and at higher amplitudes produce an onset response followed by neural block (Bhadra et al., 2018) (. 1AA). However, previous work using non-ramped KHF signals showed that onset response decreases as frequency is increased. For example, measuring KHF responses across frequencies and amplitudes showed that frequencies below 30 kHz produced responses across amplitudes consistent with the proposal by (Bhadra et al., 2018) as shown in. 1AA, whereas frequencies at or above 30 kHz produced distinct responses in which no clear ‘activation’ regime was present (. 1AB). Notably, prior investigations only considered ramped KHF signals across a limited range of ramp durations (up to 60 s) and kilohertz frequencies (up to 30 kHz) (Miles et al., 2007).

### SUMMARY

Embodiments of the present disclosure include a method for blocking neural conduction in a subject. The method includes programming a pulse generator to output an AC blocking waveform. The pulse generator is coupled to an electrode sized and configured for implantation in proximity to neural tissue in the subject. The method further includes delivering the blocking waveform to the subject without inducing an onset response. Delivering the blocking waveform includes increasing an amplitude over a ramp period of at least 15 seconds.

In some embodiments, increasing the amplitude over the ramp period includes increasing the amplitude from zero to an amplitude for blocking neural conduction over the ramp period.

In some embodiments, the AC blocking waveform has a frequency of at least 20 kHz.

In some embodiments, the AC blocking waveform has a frequency of at least 30 kHz.

In some embodiments, the AC blocking waveform has a frequency within a range of 5 kHz to 100 kHz.

In some embodiments, the ramp period is at least 100 seconds.

In some embodiments, the ramp period is within a range of 15 seconds to 600 seconds.

In some embodiments, the ramp period is adjustable and wherein a frequency of the AC blocking waveform increases as the ramp period decreases.

In some embodiments, a frequency of the AC blocking waveform is decreased after the ramp period is complete.

In some embodiments, the frequency linearly decreases after the ramp period is complete.

In some embodiments, the frequency linearly decreases from within a first range of 60 kHz to 100 kHz to within a second range of 5 kHz to 40 kHz after the ramp period is complete.

In some embodiments, the blocking waveform includes decreasing a frequency over time.

In some embodiments, the pulse generator comprises a power source comprising a battery and a microprocessor coupled to the battery.

In some embodiments, the AC blocking waveform is charge-balanced.

In some embodiments, increasing the amplitude over the ramp period is at a ramp rate, and the ramp rate is within a range of 0.55 mA/second and 0.003 mA/second.

In some embodiments, the AC blocking waveform is rectangular, sinusoidal, sawtooth, pulsed, or a sum of sinusoids.

### BRIEF DESCRIPTION OF THE DRAWINGS

FIG. 1A is an illustration of current theory of amplitude effects from KHF signals (bottom; adapted from (Bhadra et al., 2018)). The example signal (top; adapted from (Miles et al., 2007)) shows the transmission, excitation, and block of a rat sciatic nerve (with force from the calf muscle measured as proxy for the nerve activity) when ramping linearly from 0 mA to an amplitude above the minimum required to achieve block, i.e., a supra-block threshold amplitude.

FIG. 1B illustrates data collected for non-ramp KHF response measurements in a rat sciatic nerve when applying a range of amplitudes and frequencies. Colors indicate the area under the curve of a stimulus-triggered average of muscle twitches during the end of a 10-second KHF trial. At low frequencies (≤20 kHz), there was excitation (i.e., “activation”, in yellow) at the end of the KHF trial at intermediate amplitudes, and this was not evident at higher frequencies (≥30 kHz).

FIGS. 2A-2C: illustrate an overview of experimental procedures for quantifying block threshold, onset response, and the fastest ramp rate that produced nerve block without onset response at each frequency. FIG. 2A illustrates surgical preparation for tibial nerve stimulation and block. Red ‘x’ symbols indicate cut nerve branches. FIG. 2B illustrates ramp signals consisting of KHF sinusoids of specified frequencies and linearly increasing amplitudes from 0 mA to a specified maximum amplitude. FIG. 2C: (i) each trial consisted of a pre-ramp period, a ramped KHF sinusoid at a given frequency and ramp rate, a period at maximum amplitude, and a 60 sec post-KHF period; (ii) each Trial Set consisted of multiple randomized trials (i.e., multiple frequencies, multiple ramp rates); waiting ≥3 min between trials avoided short-term cumulative effects of KHF; (iii) descriptions of Trial Sets run in each animal; (iv) schematic of strategy to search for fastest onset-free ramp rate with <42% precision, where 0.05 mA/sec is used as both the initial fastest and slowest ramp rates, using the responses from Trial Set #1.

FIGS. 3A-3B: illustrate examples of ramp without (A) and with (B) onset response. FIG. 3A illustrates that muscle twitches (in red) evoked by 1 Hz test pulses decreased gradually during a 24 sec ramp at 71.4 kHz (blue). FIG. 3B illustrates that muscle twitches (red) evoked by 1 Hz test pulses also decreased gradually during a 520 sec ramp at 10 kHz (blue), but the KHF signal evoked a large onset response between 160-280 sec.

FIGS. 4A-4B: Summary of fastest ramp rates and shortest ramp durations (paired data points) that produced block without onset response across all nerves (gray). All nerves at 10 kHz and three nerves at 20 kHz had onset response at the slowest ramp rates and longest ramp durations tested (black). FIG. 4A illustrates that increasing the signal frequency allowed block without onset response to occur at faster ramp rates (i.e., lower inverse ramp rates). FIG. 4B illustrates that increasing the signal frequency allowed block without onset response to occur at shorter ramp durations. Ramp duration was defined as the time to increase the amplitude of the kilohertz-frequency signal from 0 mA to an amplitude at which test pulses produced twitch amplitudes 40% of baseline. Thick/Filled Markers: Fastest ramp with no onset response (gray; connected by lines). Thin/Unfilled Markers: Slowest ramp with onset response, shown only if all ramps for that frequency and nerve produced onset response (black; not connected by lines).

FIG. 5 illustrates examples of responses from a single nerve to KHF signals that were ramped (A) and not ramped (B) across multiple frequencies.

FIG. 6 illustrates examples of linear transition from 80 to 20 kHz (nerve ‘d’) (A) and from 60 to 40 kHz (nerve ‘e’) (B) after partially blocking the nerve without onset response at the higher frequency. The sinusoids at 80 and 20 kHz (A) and 60 and 40 kHz (B) were superposed and applied through the same electrode at the times and ramped amplitudes shown.

FIG. 7 illustrates an example of gastrocnemius force (top) in response to repeated applications of a 10 kHz square wave signals of ramped amplitude (bottom) wherein the pause between KHF applications was <30 seconds. Onset response due to the 10 kHz ramps decreased after repeated application. All KHF signals were ramped at 0.1 mA/s from 0 to 3 mA. Stimulus pulse repetition rate was 1.5 Hz.

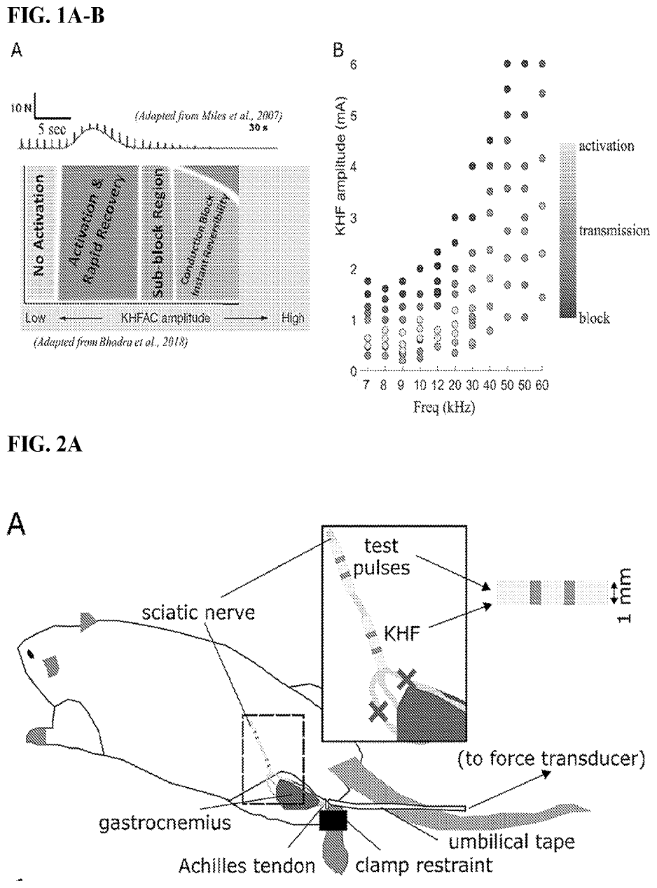

### DETAILED DESCRIPTION

The present disclosure provides systems and methods relating to neuromodulation. In particular, the present disclosure provides systems and methods for eliminating the onset response when blocking nerve conduction.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“About” is used to provide flexibility to a numerical range endpoint by providing that a given value may be “slightly above” or “slightly below” the endpoint without affecting the desired result.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Pain” generally refers to the basic bodily sensation induced by a noxious stimulus, received by bare nerve endings, characterized by physical discomfort (e.g., pricking, throbbing, aching, etc.) and typically leading to an evasive action by the individual. As used herein, the term pain also includes chronic and acute neuropathic pain. The term “chronic neuropathic pain” refers to a complex, chronic pain state that wherein the nerve fibers themselves may be damaged, dysfunctional or injured. These damaged nerve fibers send incorrect signals to other pain centers. The impact of nerve fiber injury includes a change in nerve function both at the site of injury and areas around the injury. The term “acute neuropathic pain” refers to self-limiting pain that serves a protective biological function by acting as a warning of on-going tissue damage. Acute neuropathic pain is typically a symptom of a disease process experienced in or around the injured or diseased tissue.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

The term “effective amount” or “therapeutically effective amount” refers to an amount sufficient to effect beneficial or desirable biological and/or clinical results.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. Ramped Kilohertz-frequency Blocking Waveform

One aspect of the present disclosure provides a system and method that achieves nerve block without eliciting onset response (i.e., no onset response) and without using charge imbalances. The method comprises ramping the amplitude of a kilohertz-frequency signal over a duration ranging from 15 to 1000 seconds and using a kilohertz-frequency signal of frequency ranging from 20 to 80 kHz. In some embodiments, the frequency is >10 kHz. The minimum duration needed within the range is inversely related to the frequency used within the range, such that the duration of the ramp and the frequency of the signal are tunable.

The kilohertz-frequency signal consists of a series of biphasic alternating current (AC) waveforms. These waveforms could be sinusoids, square waves, a train of rectangular pulses, sawtooth waves, or other waveform shapes. During the ramp, the amplitude of each waveform ranges from zero up to an amplitude that achieves partial or full neural block. The first waveform of the ramp has an amplitude of zero. Every subsequent waveform of the ramp has an equal or larger amplitude than the preceding waveform in the series. The last waveform of the ramp has an amplitude equal to or greater than the amplitude that produces partial or full neural block, whichever is desired. That is, the final amplitude of the blocking signal can be tuned to achieve partial or graded block. After the ramp, the kilohertz-frequency signal continues as a series of waveforms at the amplitude that produces partial or full neural block. The use of higher frequencies and longer ramp durations than prior methods enables blocking neural without onset response.

In some embodiments, the AC blocking waveform can be applied at increasing amplitude over time, which in part facilitates the elimination of the onset response. In some embodiments, the AC blocking waveform can be applied at increasing amplitude over a time period of 5 ms, 10 ms, 15 ms, 20 ms, 25 ms, 30 ms, 35 ms, 40 ms, 45 ms, 50 ms, 55 ms, 60 ms, 70 ms, 80 ms, 90 ms or 100 ms. In some embodiments, the AC blocking waveform can be applied at increasing amplitude over any time period after the initial application of the waveform (time 0) up until about 100 ms. In some embodiments, the AC blocking waveform can be applied at increasing amplitude over a time period of 5 s, 10 s, 15 s, 20 s, 25 s, 30 s, 35 s, 40 s, 45 s, 50 s, 55 s, 60 s, 70 s, 80 s, 90 s or 100 s. In some embodiments, the AC blocking waveform can be applied at increasing amplitude over any time period after the initial application of the waveform (time 0) up until at least 15 seconds.

In some embodiments, the AC blocking waveform can be applied at increasing amplitude over time at a rate within a range of approximately 0.55 mA/second and approximately 0.003 mA/second. In some embodiments, the amplitude of the AC blocking waveform is increased linearly as a function of time. In some embodiments, the amplitude is increased non-linearly as a function of time (e.g., exponentially, piecewise polynomial).

In some embodiments, the AC blocking waveform can be charge-balanced. In some embodiments, the AC blocking waveform can be biphasic, monophasic, or multiphasic. In other embodiments, the AC waveform can be multiphasic, including a waveform that is charge-balanced and/or biphasic. In some embodiments, the AC blocking waveform is symmetric and/or rectangular. In some embodiments, the AC blocking waveform can be sinusoidal. In some embodiments, the AC blocking waveform is a sawtooth. In some embodiments, the AC blocking waveform is triangular. In some embodiments, the AC blocking waveform is a sum of sinusoids.

In some embodiments, the AC blocking waveform can be applied at a frequency of at least about 5 kHz. In some embodiments, the AC blocking waveform can be applied at a frequency of at least about 10 kHz. In some embodiments, the AC blocking waveform can be applied at a frequency of at least about 20 kHz. In some embodiments, the AC blocking waveform can be applied at a frequency of at least about 30 kHz. In some embodiments, the AC blocking waveform can be applied at a frequency of at least about 40 kHz. In some embodiments, the AC blocking waveform can be applied at a frequency of at least about 60 kHz. In some embodiments, the AC blocking waveform can be applied at a frequency of at least about 80 kHz. In some embodiments, the AC blocking waveform can be applied at a frequency of at least about 90 kHz. In some embodiments, the AC blocking waveform can be applied at a frequency of about 5 kHz to about 90 kHz. In some embodiments, the AC blocking waveform can be applied at a frequency of about 5 kHz to about 80 kHz. In some embodiments, the AC blocking waveform can be applied at a frequency of about 5 kHz to about 70 kHz. In some embodiments, the AC blocking waveform can be applied at a frequency of about 5 kHz to about 60 kHz. In some embodiments, the AC blocking waveform can be applied at a frequency of about 5 kHz to about 50 kHz. In some embodiments, the AC blocking waveform can be applied at a frequency of about 5 kHz to about 40 kHz. In some embodiments, the AC blocking waveform can be applied at a frequency of about 5 kHz to about 30 kHz. In some embodiments, the AC blocking waveform can be applied at a frequency of about 5 kHz to about 20 kHz. In some embodiments, the AC blocking waveform can be applied at a frequency of about 5 kHz to about 10 kHz. In some embodiments, the AC blocking waveform can be applied at a frequency of about 10 kHz to about 90 kHz. In some embodiments, the AC blocking waveform can be applied at a frequency of about 20 kHz to about 90 kHz. In some embodiments, the AC blocking waveform can be applied at a frequency of about 30 kHz to about 90 kHz. In some embodiments, the AC blocking waveform can be applied at a frequency of about 40 kHz to about 90 kHz. In some embodiments, the AC blocking waveform can be applied at a frequency of about 50 kHz to about 90 kHz. In some embodiments, the AC blocking waveform can be applied at a frequency of about 60 kHz to about 90 kHz. In some embodiments, the AC blocking waveform can be applied at a frequency of about 70 kHz to about 90 kHz. In some embodiments, the AC blocking waveform can be applied at a frequency of about 80 kHz to about 90 kHz.

### 3. Neuromodulation Systems

Embodiments of the present disclosure also include the various AC conduction block waveforms described herein, as well as systems for blocking neural conduction without inducing an onset response in a subject using the AC blocking waveforms. In accordance with these embodiments, the AC blocking waveform can be applied at increasing amplitude over time. The application of the AC blocking waveform using these systems eliminates onset firing that often causes muscle contraction, pain, and/or other physiological effects in a subject, which is a significant impediment to potential clinical applications of KHFAC neural block.

The electrode can be one or more electrodes configured as part of the distal end of a lead or be one or more electrodes configured as part of a leadless system to apply electrical pulses to the targeted tissue region. Electrical pulses can be supplied by a pulse generator coupled to the electrode/lead. In one embodiment, the pulse generator can be implanted in a suitable location remote from the electrode/lead (e.g., in the shoulder region); however, that the pulse generator could be placed in other regions of the body or externally to the body.

When implanted, at least a portion of the case or housing of the pulse generator can serve as a reference or return electrode. Alternatively, the lead can include a reference or return electrode (comprising a multipolar (such as bipolar) arrangement), or a separate reference or return electrode can be implanted or attached elsewhere on the body (comprising a monopolar arrangement).

The pulse generator can include stimulation generation circuitry, which can include an on-board, programmable microprocessor, which has access to and/or carries embedded code. The code expresses pre-programmed rules or algorithms under which desired electrical stimulation is generated, having desirable electrical stimulation parameters that may also be calculated by the microprocessor, and distributed to the electrode(s) on the lead. According to these programmed rules, the pulse generator directs the stimulation through the lead to the electrode(s), which serve to selectively stimulate the targeted tissue region. The code may be programmed, altered or selected by a clinician to achieve the particular physiologic response desired. Additionally or alternatively to the microprocessor, stimulation generation circuitry may include discrete electrical components operative to generate electrical stimulation having desirable stimulation parameters, as described further herein (e.g., input to generate an optimized waveform shape, which can include a pulse amplitude; a pulse width (PW) or duration; a frequency of stimulation pulses applied over time; and a shape or waveform of the stimulation pulses). One or more of the parameters may be prescribed or predetermined as associated with a particular treatment regime or indication. In some embodiments, the pulse generator can be programed to output a stimulation waveform (e.g., on a graphical user interface (GUI)), and the stimulation waveform can represent a waveform having an optimized shape capable of blocking neural conduction without inducing onset response, as described further herein. In some embodiments, programming the pulse generator includes setting the amplitude of the stimulation waveform, such that the stimulation waveform blocks neural conduction when delivered by the pulse generator.

Additionally, the neuromodulation systems and methods of the present disclosure include a system for delivering neuromodulation therapy to a subject in order to reduce, treat, or prevent the subject's neuropathic pain. In accordance with these embodiments, the system includes an electrode sized and configured for implantation in proximity to neural tissue. For example, the system can include a stimulation device, an electrical connection lead, and at least one electrode or electrode array operatively positioned in the epidural space of a vertebral column of a subject that is experiencing neuropathic pain. The electrode or electrode array can be positioned at the site of neurons that are the target of stimulation (e.g., along the spinal cord), or positioned in any suitable location that allows for the delivery of electrical stimulation to the targeted neural tissue.

In some embodiments, the system includes a pulse generator coupled to the electrode. The pulse generator can include a power source comprising a battery and a microprocessor coupled to the battery, and the pulse generator is generally configured to generate electrical signals for delivering neuromodulation therapy. In some embodiments, the system further includes a controller comprising hardware, software, firmware, or combinations thereof for implementing functionality described herein. For example, the controller can be implemented by one or more processors and memory. The controller can be operatively connected to the pulse generator to facilitate the generation of electrical signals and applying temporal patterns of electrical stimulation to targeted neurological tissue. The output signals may be received by the connection lead and carried to the electrode or electrode array for the delivery of electrical stimulation to targeted neurological tissue. The system can include a power source, such as a battery, for supplying power to the controller and the pulse generator.

In some embodiments, the system also includes an external computing device that is not implanted within the subject. The computing device can communicate with a stimulation device or system via any suitable communication link (e.g., a wired, wireless, or optical communication link). The communication link may also facility battery recharge. A clinician may interact with a user interface of the computing device for programming the output of the implanted pulse generator, including the electrodes that are active, the stimulation pulse amplitude, the stimulation pulse duration, the stimulation pattern (including pulse repetition frequency), and the like applied via each electrode contact to each sub-population. In accordance with some embodiments, systems and methods of the present disclosure can be used to deliver an AC blocking waveform, as described herein, to reduce pain in a plurality of subjects with different pain states, for example, without inducing onset response. In accordance with some embodiments, systems and methods of the present disclosure can be used to deliver an AC blocking waveform, as described herein, for a number of applications including, but not limited to, treatment of heart failure, treatment of hypertension, control of bladder function, treatment for overactive bladder, control of colon motility, treatment of chronic constipation, without inducing onset response. In addition, onset free KHF block enables unidirectional neural activation or stimulation when paired with another electrode that activates neurons.

In some embodiments, systems and methods of the present disclosure can be implemented as an algorithm within a pulse generator device. An on-board controller can deliver multiple frequencies and patterns through different output channels to different contacts on an electrode, including delivering any of the AC blocking waveforms of the present disclosure. Values of the stimulation frequencies and patterns of stimulation and the electrodes through which these frequencies and patterns are delivered can be input by either a physician or a patient through a user interface. Alternatively, the device can be pre-programmed with specific combinations of frequencies and patterns to use. The applied frequencies and patterns may or may not be offset from each other at the start of stimulation. The algorithm can be toggled on and off (e.g., between multi-frequency and single frequency) by either the physician or patient, or it can be coupled to an internal feedback-driven algorithm for automatic control.

The systems described herein can be implemented in hardware, software, firmware, or combinations of hardware, software and/or firmware. In some examples, the systems described in this specification may be implemented using a non-transitory computer readable medium storing computer executable instructions that when executed by one or more processors of a computer cause the computer to perform operations. Computer readable media suitable for implementing the systems described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system described in this specification may be located on a single device or computing platform or may be distributed across multiple devices or computing platforms.

In accordance with the above, embodiments of the present disclosure include a method for blocking neural conduction in a subject. The method includes programming a pulse generator to output an AC blocking waveform. The pulse generator is coupled to an electrode sized and configured for implantation in proximity to neural tissue in the subject. The method further includes delivering the blocking waveform to the subject without inducing an onset response. Delivering the blocking waveform includes increasing an amplitude over a ramp period of at least 15 seconds.

In some embodiments, increasing the amplitude over the ramp period includes increasing the amplitude from zero to an amplitude for blocking neural conduction over the ramp period.

In some embodiments, the AC blocking waveform has a frequency of at least 20 kHz.

In some embodiments, the AC blocking waveform has a frequency of at least 30 kHz.

In some embodiments, the AC blocking waveform has a frequency within a range of 5 kHz to 100 kHz.

In some embodiments, the ramp period is at least 100 seconds.

In some embodiments, the ramp period is within a range of 15 seconds to 600 seconds.

In some embodiments, the ramp period is adjustable and wherein a frequency of the AC blocking waveform increases as the ramp period decreases.

In some embodiments, a frequency of the AC blocking waveform is decreased after the ramp period is complete.

In some embodiments, the frequency linearly decreases after the ramp period is complete.

In some embodiments, the frequency linearly decreases from within a first range of 60 kHz to 100 kHz to within a second range of 5 kHz to 40 kHz after the ramp period is complete.

In some embodiments, the blocking waveform includes decreasing a frequency over time.

In some embodiments, the pulse generator comprises a power source comprising a battery and a microprocessor coupled to the battery.

In some embodiments, the AC blocking waveform is charge-balanced.

In some embodiments, increasing the amplitude over the ramp period is at a ramp rate, and the ramp rate is within a range of 0.55 mA/second and 0.003 mA/second.

In some embodiments, the AC blocking waveform is rectangular, sinusoidal, sawtooth, pulsed, or a sum of sinusoids.

### 4. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

Examples provided herein include rat tibial nerve experiments, where nerve responses are quantified to linearly ramped KHF signals applied at a range of durations and frequencies that included longer durations (>60 s) and higher frequencies (>30 kHz) than previously considered. The ability of longer and higher frequency KHF ramps to block without onset response is evaluated, showing for the first time that the sufficiently high frequencies and sufficiently long ramp signals could block without onset response.

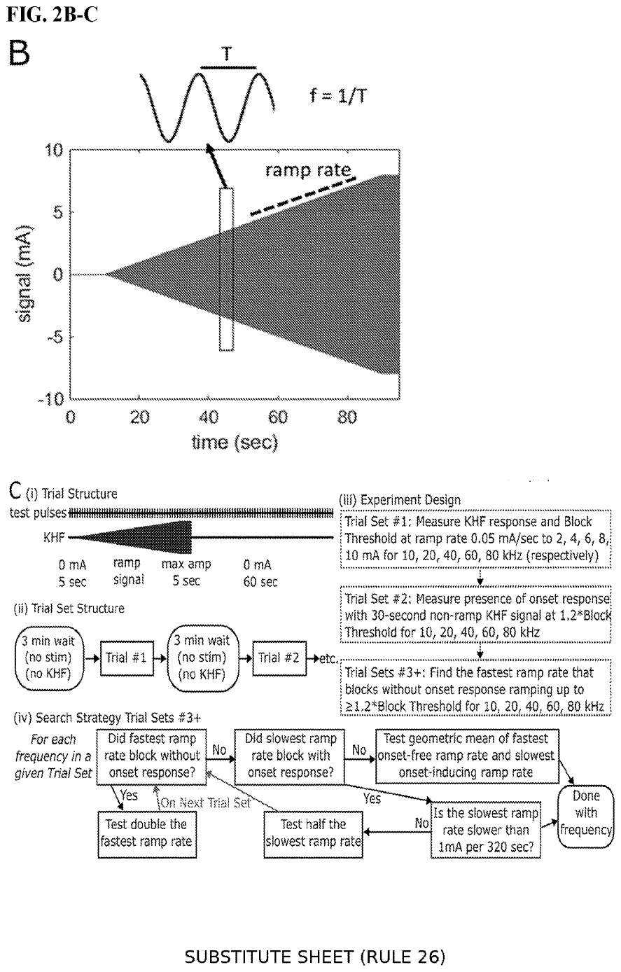

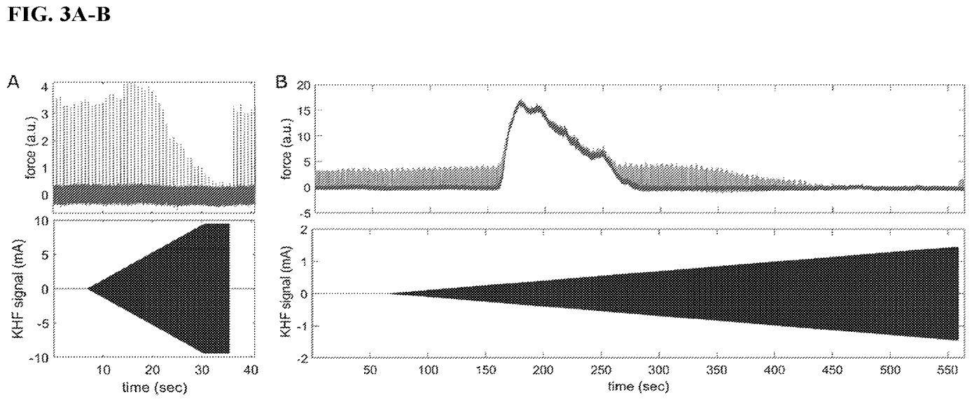

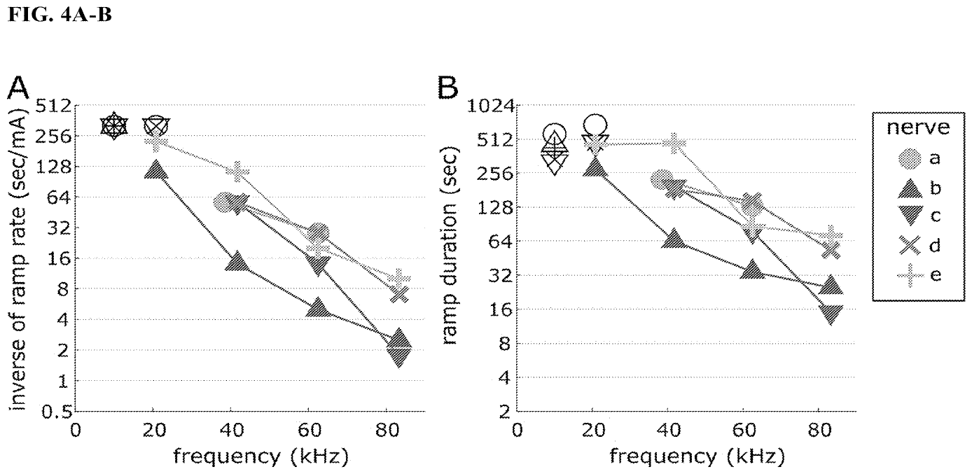

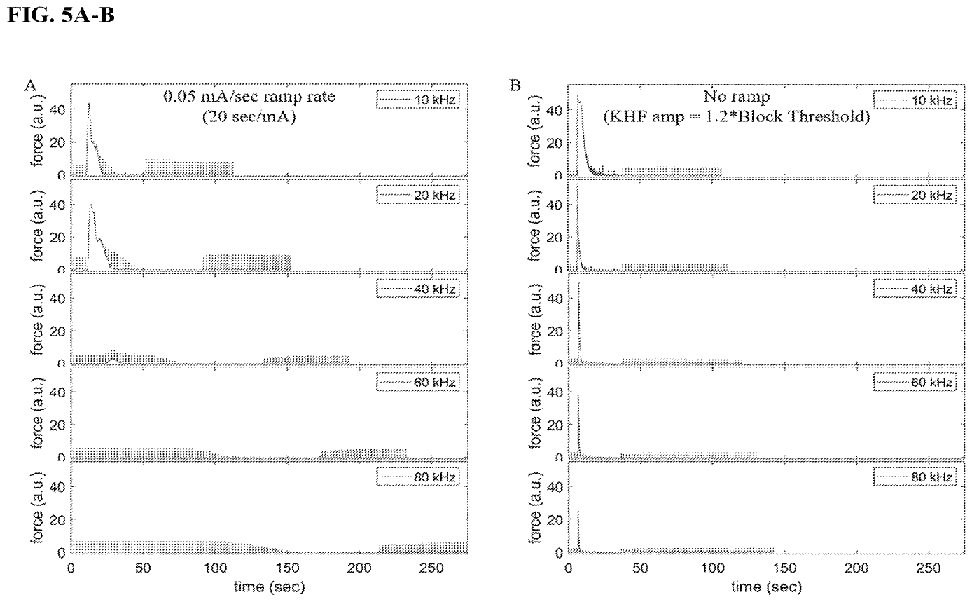

Methods. Onset response is quantified during ramped amplitude KHF signals (FIG. 2). In urethane-anesthetized rats, an electrode is placed on the proximal sciatic nerve to activate the nerve at 1 Hz and a second electrode is placed on the distal sciatic nerve to deliver KHF signals. Transmission, block, and onset responses are quantified by measuring the force of gastrocnemius contraction. The amplitude of KHF sinusoidal AC waveforms is linearly ramped from zero up to an amplitude that achieved partial or full nerve block. Multiple combinations of ramp durations (30 to 600 s) and kilohertz frequencies (10 to 80 kHz) are evaluated.

The disclosed results show block without onset response and without charge imbalance. Among frequencies that blocked without onset response, higher frequencies did so at faster ramp rates than lower frequencies. The lowest frequencies (10-20 kHz) produced onset response at all tested ramp rates. Higher frequencies generally needed shorter duration to reach 25% transmission despite higher threshold.

### Example 1

Ramped KHF signals produced nerve block without causing excitation at frequencies >10 kHz. Ramping the KHF signals for 16 to 600 s and at 20 to 80 kHz resulted in nerve block with no onset response (Error! Reference source not found. A; Error! Reference source not found.). Crucially, the maximum ramp rate or minimum ramp duration to enable block without onset response was inversely related to the blocking signal frequency (Error! Reference source not found.). Onset response was present at all ramp rates in all nerves at 10 kHz and in three nerves at 20 kHz (Error! Reference source not found.). Further, even with a 520 s ramp, 10 kHz produced a strong onset response (Error! Reference source not found. B), consistent with prior literature.

### Example 2

Non-ramped KHF signals produced onset response at all frequencies. When the signal was not ramped, onset response occurred at all frequencies (Error! Reference source not found. B). When the signal was ramped at a rate of 0.05 mA/sec, onset response only occurred at 10, 20, and 40 kHz, but not at 60 or 80 kHz (Error! Reference source not found. A).

### Example 3

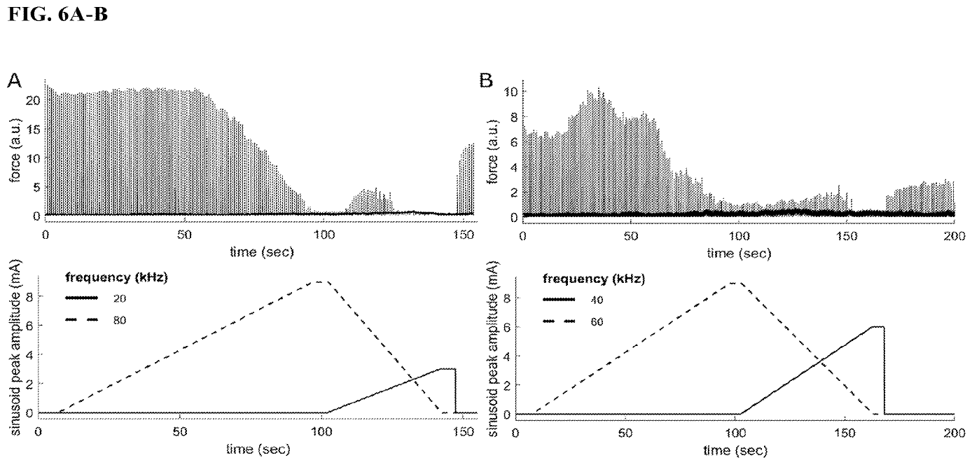

Transitioning from high frequency ramped signals to lower frequency signals enabled low frequency block without onset response. Since lower frequencies block with less energy, whether transitioning from a high frequency to a low frequency after establishing block could maintain block was investigated (FIG. 6). After partially blocking a nerve with an 80 kHz ramped signal without producing onset response, transitioning the frequency linearly down to 20 kHz enabled the nerve to be blocked without onset response. During the transition, partial block weakened such that transmitted twitches had a higher amplitude. However, twitches remained at least partially blocked, and eventually became fully blocked at sufficiently high amplitudes.

### Example 4

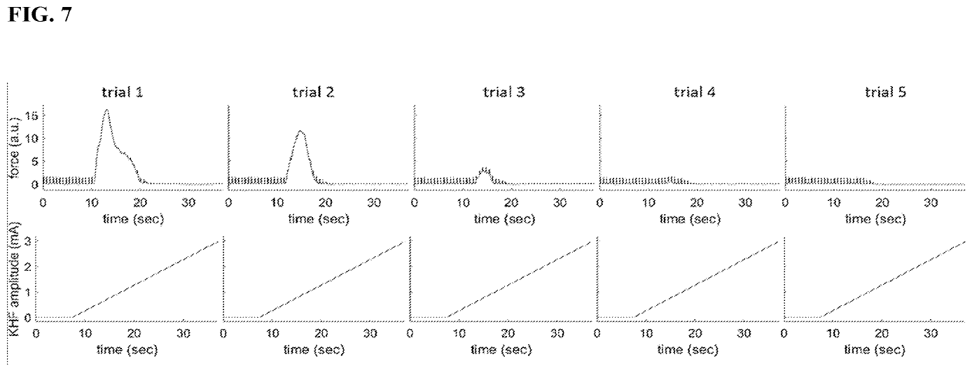

Series of Ramping Signals. With reference to FIG. 7, an example shows the effects of a series of ramping signals where the onset response is present in response to the first presentation and then declines during repeated presentation such that block is achieved in the fifth presentation with no onset response. Gastrocnemius force (top) is illustrated in response to repeated applications of a 10 kHz square wave signals of ramped amplitude (bottom) wherein the pause between KHF applications was less than 30 seconds. Onset response due to the 10 kHz ramps decreased after repeated application. All KHF signals were ramped at 0.1 mA/s from 0 to 3 mA. Stimulus pulse repetition rate was 1.5 Hz

In some embodiments, a method includes applying a series of ramping signals, with gaps in between, to achieve onset free block. In some embodiments, when applying a series of ramping signals, the signal is less than approximately 30 kHz. In some embodiments, when applying a series of ramping signals, the signal is within a range of approximately 10 kHz to approximately 30 kHz. In some embodiments, when applying a series of ramping signals, the signal is ramped over a period of less than 30 seconds. In some embodiments, when applying a series of ramping signals, the signal is ramped over a period within a range of approximately 5 seconds to approximately 30 seconds.

In some embodiments, the ramping AC blocking waveform can be applied multiple times in a sequence with a pause in between applications and at a combination of frequency and ramp rate that if applied only once produces an onset response. The pause duration between applications within the sequence of the ramping AC blocking waveforms can be tuned between 0 to 10 min to eliminate or to preserve the onset response for applications within in the sequence, wherein shortening the pause duration can eliminate the onset response on subsequent applications and wherein lengthening the pause duration can preserve the onset response on subsequent applications. Thus, repeated application of a sequence of AC blocking waveforms at a combination of frequency and ramp rate that if applied only once produces an onset response can, when applied at a sequence with sufficiently short pauses between applications, result in neural conduction block.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method for blocking neural conduction in a subject, the method comprising:
programming a pulse generator to output an AC blocking waveform; wherein the pulse generator is coupled to an electrode sized and configured for implantation in proximity to neural tissue in the subject; and
delivering the blocking waveform to the subject without inducing an onset response; wherein delivering the blocking waveform includes increasing an amplitude over a ramp period of at least 15 seconds.

2. The method of claim 1, wherein increasing the amplitude over the ramp period includes increasing the amplitude from zero to an amplitude for blocking neural conduction over the ramp period.

3. The method of claim 1, wherein the AC blocking waveform has a frequency of at least 20 kHz.

4. The method of claim 1, wherein the AC blocking waveform has a frequency of at least 30 kHz.

5. The method of claim 1, wherein the AC blocking waveform has a frequency within a range of 5 kHz to 100 kHz.

6. The method of claim 1, wherein the ramp period is at least 100 seconds.

7. The method of claim 1, wherein the ramp period is within a range of 15 seconds to 600 seconds.

8. The method of claim 1, wherein the ramp period is adjustable and wherein a frequency of the AC blocking waveform increases as the ramp period decreases.

9. The method of claim 1, wherein a frequency of the AC blocking waveform is decreased after the ramp period is complete.

10. The method of claim 9, wherein the frequency linearly decreases after the ramp period is complete.

11. The method of claim 10, wherein the frequency linearly decreases from within a first range of 70 kHz to 100 kHz to within a second range of 5 kHz to 30 kHz after the ramp period is complete.

12. The method of claim 1, wherein delivering the blocking waveform includes decreasing a frequency over time.

13. The method of claim 1, wherein the pulse generator comprises a power source comprising a battery and a microprocessor coupled to the battery.

14. The method of claim 1, wherein the AC blocking waveform is charge-balanced.

15. The method of claim 1, wherein increasing the amplitude over the ramp period is at a ramp rate, and the ramp rate is within a range of 0.55 mA/second and 0.003 mA/second.

16. The method of claim 1, wherein the AC blocking waveform is rectangular, sinusoidal, sawtooth, or pulsed.

17. The method of claim 1, wherein increasing the amplitude over the ramp period includes increasing the amplitude from zero to an amplitude for blocking neural conduction over the ramp period; and wherein the AC blocking waveform has a frequency within a range of 5 kHz to 100 kHz.

18. The method of claim 17, wherein the ramp period is within a range of 15 seconds to 600 seconds; and wherein a frequency of the AC blocking waveform is decreased after the ramp period is complete.

19. The method of claim 18, wherein increasing the amplitude over the ramp period is at a ramp rate, and the ramp rate is within a range of 0.55 mA/second and 0.003 mA/second.

20. The method of claim 19, wherein the frequency linearly decreases from within a first range of 70 kHz to 100 kHz to within a second range of 5 kHz to 30 kHz after the ramp period is complete.
